(19) United States
(12) Patent Application Publication
(10) Pub. No.: US 2026/0041725 A1
(43) Pub. Date: Feb. 12, 2026

(54) MYCONOSIDE-RICH EXTRACT FROM IN VITRO SYSTEMS OF PLANTS BELONGING TO GENERA HABERLEA AND RAMONDA, METHOD OF PREPARATION, AND USE AS CHEMO-, RADIO- AND UV PROTECTOR

(71) Applicant: INNOVA BM Ltd., Sofia (BG)

(72) Inventors: Petia Ivanova Tatarska, Sofia (BG); Vasil Georgiev Georgiev, Plovdiv (BG); Atanas Ianov Pavlov, Plovdiv (BG)

(73) Assignee: INNOVA BM Ltd., Sofia (BG)

(21) Appl. No.: 19/128,874

(22) PCT Filed: Dec. 20, 2022
(86) PCT No.: PCT/BG2022/000013

§ 371 Date: May 9, 2025

(30) Foreign Application Priority Data
Nov. 9, 2022 (BG) 113615

(51) Int. Cl. A61K 36/185; A61K 31/12; A61K 31/343; A61K 31/661; A61K 31/7034

(52) U.S. Cl. CPC A61K 36/185; A61K 31/12; A61K 31/343; A61K 31/661; A61K 31/7034; A61K 2236/333; A61K 2236/39; A61K 2236/51

(57) ABSTRACT

A myconoside containing extract is derived from in vitro systems of plants belonging to the family Gesneriaceae, in particular from the genera Haberlea and Ramonda, also including the species Haberlea rhodopensis Friv., Ramonda heldreichii (Boiss.) C. B. Clarke; Ramonda myconi (L.) Rchb.; Ramonda nathaliae Pancic & Petrovic, and Ramonda serbica Pancic, as well as from their hybrids. The extract is used in human and veterinary medicine, nutritional supplements and cosmetics as chemo-, radic- and UV protector. Extract 1 comprises 0.1 to 55.0 wt % PHE and free phenols, sugars, organic-, fatty- and amino acids, sterols up to 100 wt %, while Extract 2 comprises 55.0 to 99.9 wt % of PHE (in particular, myconoside and paucifloside), and phenolic compounds, mono-, disaccharides or their residues up to 100 wt %. Extracts 1 and 2 obtained according to this invention mainly consist of RHE (in particular, myconoside and paucifloside) within a broad spectrum and quantitative limits.

### FIELD OF THE INVENTION

The present invention relates to a myconoside containing extract derived from in vitro systems of plants belonging to the family Gesneriaceae, in particular from the genera Haberlea and Ramonda, also including the species Haberlea rhodopensis Friv., Ramonda heldreichii (Boiss.) C. B. Clarke; Ramonda myconi (L.) Rchb.; Ramonda nathaliae Pancic & Petrovic, and Ramonda serbica Pancic, as well as from their hybrids, and a method of preparation thereof. Furthermore, the present invention also relates to the use of said extract in human and veterinary medicine, nutritional supplements and cosmetics as chemo-, radicand UV protector.

### BACKGROUND OF THE INVENTION

Plants in the wild nature or those cultivated using traditional agricultural techniques often produce low levels of desired bioactive compounds. In addition, certain medicinal plants do not lend themselves to cultivation or belong to rare or endangered species, including the species belonging to the genera Haberlea and Ramonda, such as Haberlea rhodopensis, Ramonda serbica and Ramonda nathaliae, thereby making important naturally-synthesized biologically active ingredients unavailable to industry. The high demand for natural, plant-derived biologically active substances and the limitations of chemical synthesis have promoted the need to procure active ingredients by biotechnological methods.

Plant biotechnology, especially plant in vitro systems, has proved to be a promising tool for sustainable production of valuable biologically active ingredients under controlled conditions. Using plant in vitro systems, various compounds can be produced in significantly higher concentrations than in wild plants without damage to natural habitats and disruption of biodiversity.

In recent years, plants belonging to the Gesneriaceae family and plant species such as Haberlea rhodopensis and Ramonda serbica have attracted the attention of researchers with their beneficial effect on human and animal health.

In a joint study, national in vitro collections of Ramonda serbica in Albania and Bulgaria, and of Ramonda nathaliae in Macedonia were established, and a method of micropropagation was chosen with assessment of polymorphism of some natural populations (E. Daskalova et. al, April 2012, Biotechnology & Biotechnological Equipment 26(1):16-25).

Metabolic changes in Ramonda serbica and Ramonda nathaliae have been investigated during the processes of their desiccation and recovery by means of 1 H-NMR and GC-MS, and the metabolic composition of the plants has been published (Dejan Godeva, et. al, Phytochemical analysis vol 33, issue 6/p. 961-970, August 2022).

Studies of classical hydroalcoholic extracts of Haberlea rhodopensis have revealed the unique medicinal properties and pharmaceutical potential related ‘to their antioxidant, radioprotective, anticlastogenic, chemoprotective, cytoprotective, antimicrobial, antimutagenic, immunological, anticancer, and anti-aging effects (Bankova R., et al., Tradition and modernity in veterinary medicine, 2022, vol. 7, No. 1(12).

It has been confirmed in literature that increased free radicals (ROS) in cells—the so-called oxidative stress—is implicated in genomic DNA damage and the occurrence of mutations. ROS resulting from oxidation-reduction reactions in the body are highly reactive, can cause significant biological damage, leading to mutations in key genes and ultimately to the development of cancer, chronic diseases, etc.

The antioxidant properties of extracts from intact plants, especially of phenolic components from Haberlea rhodopensis, are known from literature (Kondeva-Burdina M, et al., Pharmacogn Mag. 2013 October; 9(36)) and (Mihaylova, D. Et al., V., 2013. J. F. Biochemistry, 37(3). Numerous studies have demonstrated that modern man is exposed to environmental factors such as solar radiation (UV radiation in the range of the UVA and UVB bands).

Moreover, free radicals generated after exposure to gamma radiation are highly reactive and can trigger chain reactions leading to significant biological damage.

Anticancer chemotherapy also induces strong oxidative stress that affects multiple cellular targets and can reduce the action of anticancer drugs. The usage of antioxidant supplements during chemotherapy can enhance the therapeutic effect (Conklin K A. Chemotherapy-associated oxidative stress: impact on chemotherapeutic effectiveness. Integr Cancer Ther. 2004 December; 3(4).

Extracts from intact Haberlea rhodopensis plants possess radioprotective properties (Georgieva S, et. al., Indian J. Exp. Biol. 2013 51(1), Georgiev, Y. N. et al., J. Ethnopharmacology, 2020, 249; Bankova, R., 2022, Tradition and modernity in veterinary medicine, 2022, vol. 7, No 1(12); Georgieva, S., Gencheva, D., Popov, B., Grozeva, N., & Zhelyazkova, M. 2019, Radioprotective action of resurrection plant Haberlea rhodopensis Friv. (Gesneriaceae) and role of flavonoids and phenolic acids, Bulgarian Journal of Agricultural Science, 25(Suppl. 3), 158-168, however, the compounds exhibiting such properties have not been studied, but have been suggested to be due to the presence of phenolic acids and flavonoids in the extract.

It is also known that certain naturally glycosylated polyphenols (GPPs) (mainly verbascoside) exhibit sunscreen properties (US 2017/16613858).

An extract from another type of cell culture belonging to the genera Syringa, in particular from Syringa vulgaris, is described in patent EP2319914 with a method of its preparation. The extract described comprises 20 to 90 wt % of phenylpropanoids (which contain from 5-20% isoverbascoside), and 80 to 10 wt % of other chromophore-free fractions, containing mainly oligo- and polysaccharides, proteins and lipid molecules. This cell culture has high antioxidant collagen-stimulating activity, pigmentation-regulating activity and characteristics.

WO2021184086 (Innova BM) describes an extract from in vitro cultures of Haberlea rhodopensis, comprising 25-35 wt % of polyphenols of the total extract, and other fractions up to 100 wt %, such as organic-, fatty- and amino acids, together with sterols, sugars and free phenols, with the phenylethanoid myconoside accounting for 18 to 35 wt % of the polyphenolic fraction. The extract described contains a limited amount of polyphenols, respectively phenylethanoid glycosides such as myconoside. The cited document describes in detail the principles of developing plant in vitro systems and the propagation of plant in vitro cultures on solid and in liquid culture media. The specific conditions used therein for large-scale cultivation of plant in vitro systems are appropriate for the specific plant species and are essential for the production of specific biologically active compounds/metabolites, as well as for target compounds in controlled quantities, and their utilization for different purposes. The broad range of controlled content of extracts from plant in vitro systems used as natural chemo-, radio- and UV protectors with high efficiency and low toxicity is particularly important as it makes possible their use in products with different applications, fine dosing at different levels of irradiation, and, therefore, rapid elimination of the harmful genotoxic effects of irradiation.

The problem of the present invention to be solved is to find an extract with controlled content of rare phenylethanoids (specifically myconoside and paucifloside), derived from in vitro systems of plants belonging to the genera Haberlea and Ramonda of the Gesneriaceae family, as well as from their hybrids, which extract has a high biological value and will be used in new products with guaranteed and controlled chemo-, radio- and UV protective effects.

### BRIEF DESCRIPTION OF THE DRAWINGS

FIG. 1 is a chart illustrating GC-MS profiles of extracts from in vitro systems of Haberlea rhodopensis obtained according to Example 1 of the present disclosure.

FIG. 2 is a graph illustrating UV spectra of myconoside solutions obtained according to Example 4 of the present disclosure.

FIG. 3 is a graph illustrating UVA and UVB light transmission for dilutions of an extract obtained according to Example 4 of the present disclosure.

FIG. 4 is a graph illustrating a change in the percentage of trapped DPPH radicals in an extract obtained according to Example 5 of the present disclosure.

FIG. 5 is a graph of DPPH radical scavenging activity (% inhibition) of aqueous solutions of extracts obtained according to Example 6 of the present disclosure.

FIG. 6 is a graph illustrating the radioprotective effect of extracts obtained according to Example 6 of the present disclosure before and after gamma irradiation.

FIG. 7A is a chart showing the number of MN found in the examination of 500 binuclear cells in cell cultures prepared with non-irradiated blood according to Example 7 of the present disclosure.

FIG. 7B is a chart showing the number of MN found in the examination of 500 binuclear cells in the cell cultures prepared with irradiated blood according to Example 7 of the present disclosure.

FIG. 8A is a chart showing the frequency of aberrant cells in human lymphocytes treated according to Example 8 of the present disclosure before gamma irradiation

FIG. 8B is a chart showing the frequency of aberrant cells in human lymphocytes treated according to Example 8 of the present disclosure after gamma irradiation.

### DESCRIPTION OF THE INVENTION

According to the present invention, this problem is solved by a myconoside containing extract derived from in vitro systems of plants belonging to the genera Haberlea and Ramonda of the Gesneriaceae family, in particular, from the species Haberlea rhodopensis Friv., Ramonda heldreichii (Boiss.) C. B. Clarke; Ramonda myconi (L.) Rchb.; Ramonda nathaliae Pancic & Petrovic and Ramonda serbica Pancic, and from their hybrids, comprising, alone or in combination, Extract 1 of polyphenolic fraction, containing mainly flavone-C glycosides and phenylethanoids (PHE), and Extract 2 of myconoside-rich fraction, where Extract 1 comprises 0.1 to 55.0 wt % PHE (in particular, myconoside and paucifloside) and free phenols, sugars, organic-, fatty- and amino acids, sterols up to 100 wt %, while Extract 2 comprises 55.0 to 99.9 wt % of PHE (in particular, myconoside and paucifloside), and phenolic compounds, mono-, disaccharides or their residues up to 100 wt %.

Another subject of the present invention is a method of preparing said Extract from the plant in vitro systems mentioned above, according to the invention, comprising the following stages:
a) Subjecting biomass (containing 0.01-25.0% phenylethanoid glycosides) obtained after initiation of the in vitro cultures and their cultivation (conducted according to WO2021184086) to aqueous or aqueous alcoholic extraction (C1-C3, 40-90%, e.g. ethanol, isopropanol, methanol) in a ratio of 1:10 to 1:100 w/v, and subsequent single- or up to three-stage extractions of the aqueous residue with a non-polar solvent approved for use in the food, pharmaceutical and cosmetic industries, until maximum removal of fat-soluble fractions; b) The purified aqueous phase from stage a) is vacuum concentrated to ⅔ of its volume, at a temperature of 40° C.-60° C. to remove the solvent, until a crude extract is obtained, which is subjected to solid-phase extraction with a hydrophobic interaction resin (in particular C18 reversed-phase) to retain phenolic compounds and RHE, while sugars and polar compounds (mainly organic and amino acids) are washed away with the aqueous mobile phase; c) Elution of the phenolic fraction from the resin by elution with mobile phase chosen from among solvents of different polarity (in particular, ethyl acetate or isopropyl alcohol). The resulting fraction is evaporated to dryness under vacuum at a temperature of 20° C.-70° C. to obtain Extract 1 comprising 0.1 to 55.0 wt % of PHE (in particular, myconoside and paucifloside), and free phenols, sugars, organic-, fatty- and amino acids up to 100 wt %; d) Elution of the PHE fraction from the resin by elution with an aqueous alcohol (C1-C3) mixture, 5 to 70% v/v. The collected PHE fraction is subjected to concentration under vacuum at a temperature of 40° C. to 60° C. until complete removal of the organic solvent, followed by lyophilization or drying to obtain Extract 2 comprising 55.0 to 99.9 wt % of PHE (in particular, myconoside and paucifloside), and phenolic compounds, mono-, disaccharides or their residues up to 100 wt %.

The resulting Extract 1 and Extract 2 can be used alone and in combination by metered blending, and conditioned by diluting with a solvent that is safe to use the in food, cosmetics and pharmaceutical industries (e.g. water, glycerin, propylene glycol or butyl glycol) until desired final concentrations of phenylethanoid glycosides (myconoside and paucifloside) is reached, in particular, standardization to a final concentration of myconoside of 0.001% to 99.0%. The product thus obtained is ready to use and provides controlled content of biologically active substances.

Another further subject of the present invention is the separate or combined use of said Extract 1 and Extract 2, derived from plant in vitro systems of plants belonging to the family Gesneriaceae, in particular from the genera Haberlea and Ramonda, specifically from the species Haberlea rhodopensis Friv., Ramonda heldreichii (Boiss.) C. B. Clarke; Ramonda myconi (L.) Rchb.; Ramonda nathaliae Pancic & Petrovic, and Ramonda serbica Pancic, as well as from their hybrids, directly or after conditioning, to obtain new agents used in human and veterinary medicine, nutritional supplements and cosmetics as chemo-, radio- and UV protectors.

The possibility of metered blending of Extract 1 and Extract 2 ensures feasibility of the specific needs and purposes of use. At final concentrations of phenylethanoid glycosides (in particular, myconoside and paucifloside) of 0.01 to 99.9%, the product thus obtained can be used as purified extract from in vitro cultures of plants of the genera Haberlea and Ramonda, with biological activity significantly higher compared to the unpurified crude extract.

Extracts 1 and 2 obtained according to this invention mainly consist of PHE (in particular, myconoside and paucifloside) within a broad spectrum and quantitative limits. These compounds (in particular, myconoside) possess high biological activities, antioxidant, antimutagenic and anticlastogenic activities proven herein, therefore are suitable for use alone and in combination by metered blending, as well as to be conditioned by dilution, for the preparation of products used as natural chemo-, radio- and UV protectors with a controlled degree of protection. The latter are used in human and veterinary medicine, nutritional supplements and cosmetics for the prevention and treatment of harmful chemo-, radio- and UV effects.

### Definitions

According to the method of the present invention, “Extract means a fraction comprising the phenylethanoid Myconoside, obtained by direct extraction or fractionation of crude extract derived from in vitro systems of plants belonging to the Gesneriaceae family, in particular from the genera Haberlea and Ramonda, as well as from their hybrids.

As used herein, the term “Plant refers to members of the family Gesneriaceae, in particular the genera Haberlea and Ramonda, specifically the species Haberlea rhodopensis Friv., Ramonda heldreichii (Boiss.) C. B. Clarke; Ramonda myconi (L.) Rchb.; Ramonda nathaliae Pancic & Petrovic, and Ramonda serbica Pancic, and their hybrids, and refers to plant cells, whole plants, plant organs, plant tissues, seeds, and progeny thereof.

As used herein, the term “In vitro Systems” refers without limitation to cells from seeds, embryos, meristematic regions, leaves, roots, shoots, gametophytes, sporophytes, pollen and microspores, callus tissue, suspension cultures, sprouts, meristem (shoot) cultures, root cultures (normal, supplementary and transformed roots) as well as in vitro plants cultivated under controlled in vitro conditions in solid or liquid nutrient media or cultivation substrate. “Myconoside” is a caffeoyl phenylethanoid glycoside with molecular formula: C33H44019, IUPAC Name: “[(2R,3R,4R,5R,6R)-4-[(2R,3R,4R)-3,4-dihydroxy-4-(hydroxymethyl)oxolan-2-yl]oxy-2-[[(2R,3R,4R)-3,4-dihydroxy-4-(hydroxymethyl)oxolan-2-yl]oxymethyl]-6-[2-(3,4-dihydroxyphenyl)ethoxy]-5-hydroxyoxan-3-yl]3-(3,4-dihydroxyphenyl)propanoate”

### EXEMPLARY EMBODIMENTS OF THE INVENTION

The invention is illustrated, but is not limited, by the following exemplary embodiments:

### Example 1

1. 500 grams of dry biomass from an in vitro culture of Haberlea rhodopensis (comprising 12% phenylethanoid glycosides) is extracted with a water-alcohol mixture (70% alcohol such as ethanol, in a ratio of 1:15 w/v). After filtration, the liquid phase is concentrated under vacuum until the alcohol is removed, then subjected to triple (3×2 L., for 1 hour, at 21° C.) liquid-liquid extraction with purified hexane to remove fat-soluble fractions. The purified aqueous phase is concentrated to ⅔ of its volume under vacuum at a temperature of 40° C. to remove solvent residues, whereby a crude extract is obtained and subjected to solid phase extraction;
2. The crude extract (2.0 L) is passed through solid phase extraction using conditioned C18 reversed phase resin (600 g). Thereby, phenolic compounds and phenylethanoid glycosides are retained by the resin, and sugars and polar compounds (mainly organic and amino acids) are washed away with the aqueous mobile phase;
3. The next step is elution of the phenolic fraction using ethyl acetate (4 L) as mobile phase, whereby most of the phenolic compounds and flavonoids as well as some of the PHE (in particular, myconoside and paucifloside) are eluted. The resulting fraction is evaporated to dryness under vacuum at a temperature of 40° C. to obtain Extract 1 comprising 54.7 wt % of PHE (in particular, myconoside and paucifloside) and free phenols, sugars, organic-, fatty- and amino acids up to 100 wt % (See Table 1 for differences in the concentrations of concomitant metabolites in various extracts, in GC-MS profiles of extracts from in vitro systems of Haberlea rhodopensis: Crude extract, Extract 1, and Extract 2. Results were statistically processed and clustered by the Euclidean distance method and are presented as Hierarchical Clustering Heatmaps, with each colored cell on the map corresponding to a concentration value of the relevant compound.
4. The next step is elution of the phenylethanoid-glycoside fraction using 2 L of water-ethanol mixture (70%, w/w). The collected PHE fraction contains mainly myconoside, and a lesser amount of paucifloside. This fraction is concentrated on a vacuum evaporator at a temperature of 50° C. until a gummy mass (80 g) is formed, which is lyophilized or dried at a temperature of 40° C. to obtain Extract 2, comprising 99.5 wt % of PHE (in particular, myconoside and paucifloside—Table 2), and mono-, disaccharides, phenolic compounds, flavonoids or flavonoid residues up to 100 wt % (Table 1).

The yield of myconoside is 76.1% as compared to its content in the original crude extract.

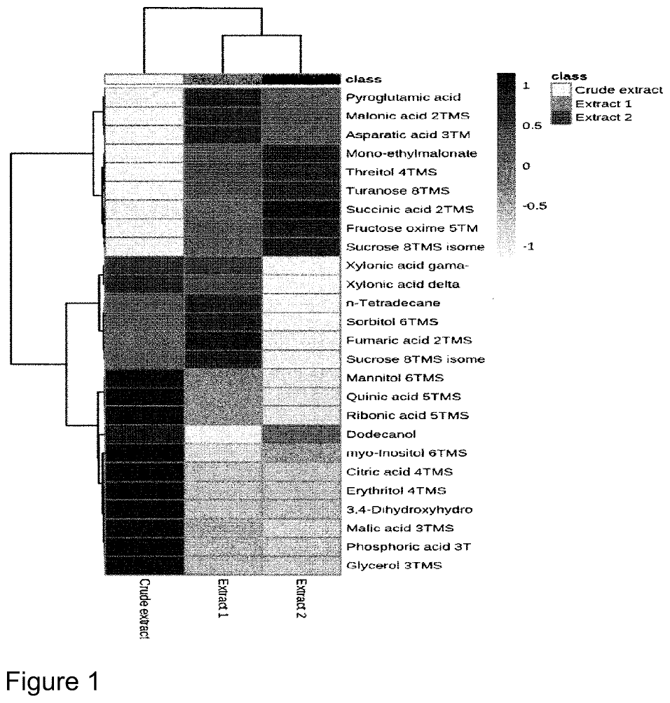

FIG. 1 shows GC-MS profiles of extracts from in vitro systems of Haberlea rhodopensis: Crude Extract, Extract 1, and Extract 2, obtained according to Example 1. The results were statistically processed and clustered by the Euclidean Distance method, and are presented as Hierarchical Clustering Heatmaps, with each colored cell on the map corresponding to a concentration value of the relevant compound.

Table 1 (below) presents the phytochemical composition of extracts from in vitro systems of Haberlea plants, obtained by the method described in Example 1 of the present invention. The results are averages of 3 parallel tests for each extract variant and presented as % of dry mass.

TABLE 1

| Metabolites | Crude extract | Extract 1 | Extract 2 |
|---|---|---|---|
| Myconoside | 7.57 | 3.03 | 72.29 |
| Phenolics and | 8.64 | 45.14 | 7.65 |
| flavone C-glycosides | | | |
| Organic acids | 72.31 | 47.21 | 16.94 |
| and saccharides | | | |
| Hydrocarbons and | 3.38 | 1.04 | 0.09 |
| fatty alcohols | | | |
| Amino acids | 2.67 | 0.38 | 0.15 |
| Others | 5.43 | 3.2 | 2.88 |

Agilent Technology Hewlett Packard 7890 A+/MSD 5975 apparatus (Hewlett Packard, Palo Alto, CA, US) coupled with an Agilent Technology 5975C inert XL EI/CI MSD Mass Spectrometer (Hewlett Packard, Palo Alto, CA, US). HP-5MS column (30 m×250 pm×0.25 pm) at 60° C. temperature program for 2 min, with a temperature rise to 260° C. with 5° C. per minute, and exposure at 260° C. for 8 min. The volume of the injected sample is 1 μl at a split ratio of 10:1. Injector temperature 250° C. with a flow carrying gas (helium) of 1 mL/min. The EI/MS spectrum is recorded at 70 eV.

HPLC system, Waters 1525 Binary pump (Waters, Milford, MA, USA), Waters 2487 Dual A Absorbance Detector (Waters, Milford, MA, USA) operated by Breeze 3.30 software; Supelco Discovery HS C18 column (5 μm, 25 cm×4.6 mm) t 28° C.; mobile phase with a gradient of 2% acetic acid and acetonitrile;

Table 2 shows the NMR data demonstrating the structures of myconoside and paucifloside (500 MHz, in D20) in Extract 2 of the present invention Table 2

TABLE 2

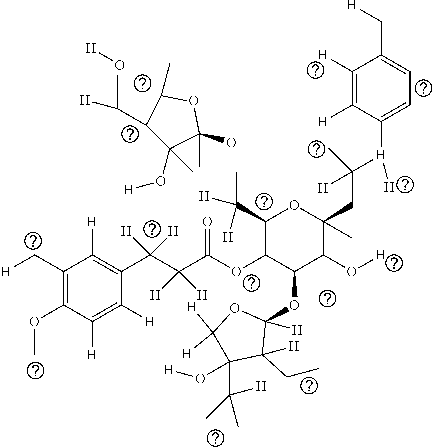

Myconoside

| No | Atom Type | δ H (Multiplicity, J) | δ C |
|---|---|---|---|
| 20 | C | | 79.39 |
| 21 | C | | 71.04 |
| 22 | C | | 73.51 |
| 23 | C | | 73.51 |
| 24 | C | | 110.15 |
| 25 | C | | 79.39 |
| 26 | C | | 76.98 |
| 27 | C | | 101.84 |
| 28 | C | | 68.98 |
| 29 | C | | 73.51 |
| 30 | C | | 79.39 |
| 31 | C | | 76.98 |
| 32 | C | | 110.15 |
| 33 | C | | 65.96 |
| 34 | C | | 73.51 |
| 35 | C | | 65.96 |
| 36 | C | | 71.04 |
| 37 | C | | 174.42 |
| 38 | C | | 34.42 |
| 39 | C | | 35.22 |
| 40 | C | | 34.42 |
| 41 | C | | 131.37 |
| 42 | C | | 133.10 |
| 43 | C | | 116.66 |
| 44 | C | | 121.14 |
| 45 | C | | 116.17 |
| 46 | C | | 120.62 |
| 47 | C | | 143.92 |
| 48 | C | | 116.66 |
| 49 | C | | 143.92 |
| 50 | C | | 143.92 |
| 51 | C | | 116.17 |
| 52 | C | | 143.92 |
| 53 | H | 4.03 (dt, 10.1, 6.8 Hz) | |
| 54 | H | 4.03 (dt, 10.1, 6.8 Hz) | |
| 55 | H | 3.86 (m) | |
| 56 | H | 3.86 (m) | |
| 57 | H | 6.74 (ddd, 10.6, 8.1, 2.0 Hz) | |
| 58 | H | 3.95 (m) | |
| 59 | H | 3.95 (m) | |
| 60 | H | 3.60 (m) | |
| 61 | H | 3.86 (m) | |
| 62 | H | 3.60 (m) | |
| 63 | H | 3.86 (m) | |
| 64 | H | 3.95 (m) | |
| 65 | H | 3.95 (m) | |
| 66 | H | 3.60 (m) | |
| 67 | H | 3.60 (m) | |
| 68 | H | 3.60 (m) | |
| 69 | H | 3.86 (m) | |
| 70 | H | — | |
| 71 | H | 3.60 (m) | |
| 72 | H | 3.60 (m) | |
| 73 | H | — | |
| 74 | H | — | |
| 75 | H | 3.86 (m) | |
| 76 | H | 3.86 (m) | |
| 77 | H | — | |
| 78 | H | — | |
| 79 | H | — | |
| 80 | H | 2.83 (m) | |
| 81 | H | 2.83 (m) | |
| 82 | H | 2.83 (m) | |
| 83 | H | 2.83 (m) | |
| 84 | H | — | |
| 85 | H | 2.83 (m) | |
| 86 | H | 2.83 (m) | |
| 87 | H | 6.74 (ddd, | |
| | | 10.6,8.1,2.0 Hz) | |
| 88 | H | 6.74 (ddd, | |
| | | 10.6,8.1,2.0 Hz) | |
| 89 | H | 6.74 (ddd, | |
| | | 10.6,8.1,2.0 Hz) | |
| 90 | H | 6.74 (ddd, | |
| | | 10.6,8.1,2.0 Hz) | |
| 91 | H | 6.74 (ddd, | |
| | | 10.6,8.1,2.0 Hz) | |
| 92 | H | 6.74 (ddd, | |
| | | 10.6,8.1,2.0 Hz) | |
| 93 | H | — | |
| 94 | H | — | |
| 95 | H | — | |
| 96 | H | — | |

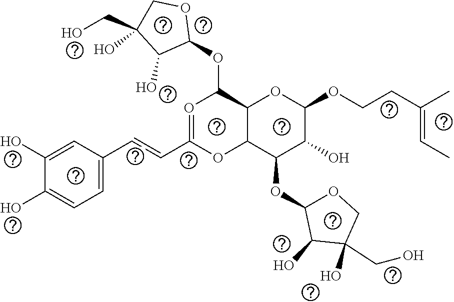

Paucifloside

| No | Atom Type | δ H (Multiplicity, J) | δ C |
|---|---|---|---|
| 1 | C | | 168.15 |
| 2 | C | | 116.17 |
| 3 | C | | 143.84 |
| 6 | C | | 131.36 |
| 7 | C | | 116.17 |
| 8 | C | | 143.84 |
| 9 | C | | 143.84 |
| 10 | C | | 116.66 |
| 11 | C | | 122.87 |
| 15 | C | | 73.64 |
| 16 | C | | 71.05 |
| 17 | C | | 79.22 |
| 18 | C | | 73.64 |
| 19 | C | | 101.85 |
| 22 | C | | 71.05 |
| 23 | C | | 35.22 |
| 24 | C | | 131.36 |
| 25 | C | | 116.66 |
| 26 | C | | 143.84 |
| 27 | C | | 143.84 |
| 28 | C | | 116.66 |
| 29 | C | | 122.87 |
| 32 | C | | 68.98 |
| 35 | C | | 110.15 |
| 36 | C | | 77.05 |
| 37 | C | | 79.44 |
| 38 | C | | 73.64 |
| 39 | C | | 65.97 |
| 45 | C | | 110.15 |
| 46 | C | | 77.05 |
| 47 | C | | 79.44 |
| 48 | C | | 73.64 |
| 49 | C | | 65.97 |
| 2 | CH | 6.74 (t, 9.5 Hz) | |
| 3 | CH | 6.87 | |
| 7 | CH | 6.87 | |
| 10 | CH | 6.74 (t, 9.5 Hz) | |
| 11 | CH | 6.74 (t, 9.5 Hz) | |
| 12 | OH | — | |
| 13 | OH | — | |
| 15 | CH | 3.86 | |
| 16 | CH | 3.86 | |
| 17 | CH | 3.86 | |
| 18 | CH | 3.86 | |
| 19 | CH | 3.95 | |
| 20 | OH | — | |
| 22 | CH2 | 3.86 | |
| 23 | CH2 | 3.59 (m) | |
| 25 | CH | 6.74 (t, 9.5 Hz) | |
| 28 | CH | 6.74 (t, 9.5 Hz) | |
| 29 | CH | 6.74 (t, 9.5 Hz) | |
| 30 | OH | — | |
| 31 | OH | — | |
| 32 | CH2 | 3.86 | |
| 35 | CH | 5.20 (s) | |
| 36 | CH | 3.95 | |
| 38 | CH2 | 3.86 | |
| 39 | CH2 | 3.59 (m) | |
| 40 | OH | — | |
| 41 | OH | — | |
| 42 | OH | — | |
| 45 | CH | 5.20 (s) | |
| 46 | CH | 3.95 | |
| 48 | CH2 | 3.86 | |
| 49 | CH2 | 3.86 | |
| 50 | OH | — | |
| 51 | OH | — | |
| 52 | OH | — | |

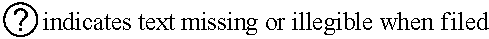

1. 100 g of dry biomass from an in vitro culture of Ramonda serbica (containing 0.5% phenylethanoid glycosides, mainly myconoside) is extracted with distilled water in a ratio of 1:30, w/v. After filtration, the liquid phase is concentrated under vacuum to ⅓ of its volume, and is thereafter subjected to triple (3×2 L., for 1 hour, at 21° C.) liquid-liquid extraction with ethyl acetate to remove fat-soluble fractions. The purified aqueous phase is concentrated to ⅔ of its volume under vacuum at a temperature of 50° C. to remove solvent residues, whereby a crude extract is obtained and subjected to solid-phase extraction according to the procedure described in points 2, 3 and 4 of Example 1 above.
2. The obtained phenolic fraction (Extract 1) contains 2.0 wt % of PHE (in particular, myconoside and paucifloside) and free phenols, sugars, organic-, fatty- and amino acids up to 100 wt %.
3. The resulting phenylethanoid-glycoside fraction (Extract 2) comprises 57.0 wt % of PHE (in particular, myconoside and paucifloside), and mono-, disaccharides and phenolic compounds and flavonoids or flavonoid residues up to 100 wt %.

The yield of myconoside is 82% compared to its content in the original crude extract.

### Example 3

It is carried out as in Example 1, with the difference that the biomass used from the in vitro culture of Haberlea rhodopensis contains 9% phenylethanoid glycosides in Step 1 and is extracted three times with 50% isopropanol at a ratio of 1:20, with the concentration of the resulting purified aqueous phase carried out at 50° C., and elution of the phenylethanoid-glycoside fraction in Step 4 carried out with 50% v/v methanol. Extract 1 thus obtained contains 29.5% PHE, specifically myconoside and paucifloside, and Extract 2 contains 62.0% PHE, specifically myconoside and paucifloside.

The yield of myconoside is 92% as compared to its content in the original crude extract.

### Example 3.a

It is conducted as in Example 3, with the difference that the dry biomass is from an in vitro culture of Ramonda serbica (containing 12% phenylethanoid glycosides, mainly myconoside). The resulting Extract 1 contains 52.0% PHE, specifically myconoside and paucifloside, and Extract 2 contains 98.0% PHE, specifically myconoside and paucifloside. The yield of myconoside is 84% as compared to its content in the original crude extract.

Table 3 (below) presents the phytochemical composition of extracts from in vitro systems of Ramonda serbica plants obtained by the method described in Example 3a of this invention. The results are averages of 3 parallel tests for each extract variant and presented as % of dry mass.

TABLE 3

| Metabolites | Crude extract | Extract 1 | Extract 2 |
|---|---|---|---|
| Myconoside | 7.07 | 2.53 | 71.19 |
| Phenolics and | 9.14 | 44.64 | 8.75 |
| flavone C-glycosides | | | |
| Organic acids | 71.31 | 46.21 | 16.04 |
| and saccharides | | | |
| Hydrocarbons and | 4.38 | 2.04 | 0.99 |
| fatty alcohols | | | |
| Amino acids | 3.67 | 0.28 | 0.10 |
| Others | 4.43 | 3.4 | 2.93 |

Extract 1 and Extract 2 obtained according to the above examples are used alone or in combination, in a dry state or conditioned by diluting with a solvent that is safe for use in food, cosmetics and pharmaceuticals (e.g. water, glycerin, propylene glycol, butyl glycol, etc.) to final concentrations of phenylethanoid glycosides (in particular, myconoside and paucifloside) of 0.01 to 99.0%, and the product thus obtained is used as a purified extract of in vitro cultures of Haberlea rhodopensis and Ramonda with significantly increased biological activity.

### EXAMPLES OF USE OF THE EXTRACTS ACCORDING TO THE INVENTION AND DATA ON THEIR EFFECTS

### Materials and Method

In this invention, Extract 1 and Extract 2, containing phenylethanoid glycosides (in particular, myconoside and paucifloside), are isolated from in vitro systems of plants belonging to the family Gesneriaceae, in particular from the genera Haberlea and Ramonda, as well as from their hybrids. Dry extracts are stored at −20° C. away from light and moisture. The solution used in the experiments is prepared extempore on the basis of an aqueous solution.

### Example 4

Study of the UV-VIS Absorption Spectrum of Extracts 1 and 2 Obtained from In Vitro Systems from Haberlea rhodopensis According to Example 1.

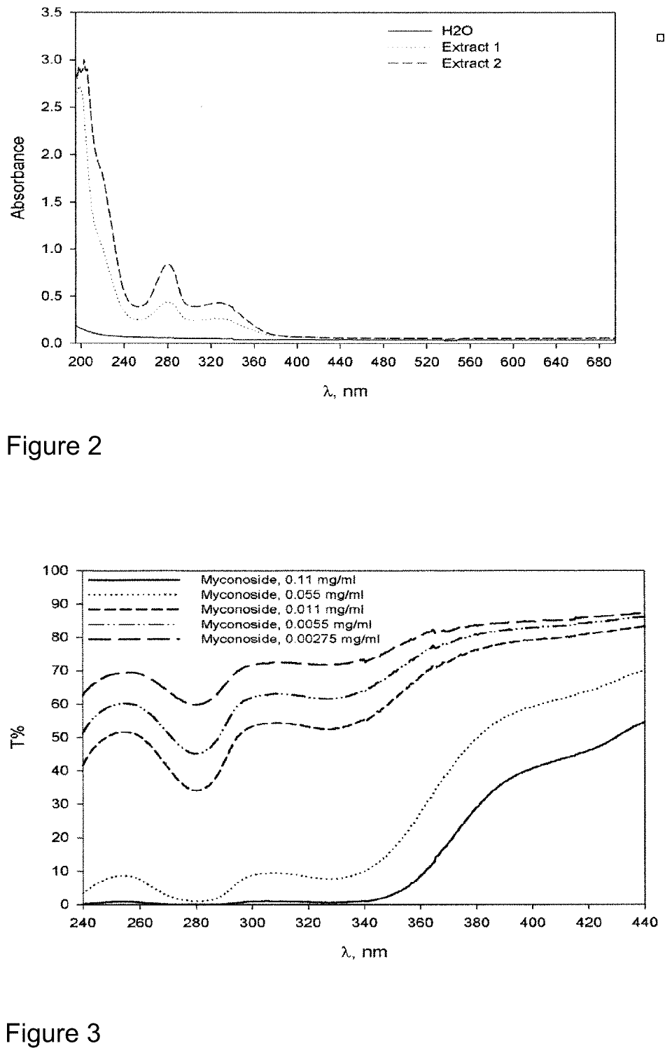

The investigated extracts are used to prepare aqueous solutions with a final myconoside concentration of 22 pM, whose UV-Vis spectra are recorded on a Shimadzu UV/Vis mini 1240 spectrophotometer. The resulting spectra are presented in FIG. 2 and demonstrate that Extract 2 has a significantly higher ability to absorb light from the UV spectrum, which is evidence that the purified myconoside has a more pronounced ability to capture light from the UV spectrum compared to the less purified Extract 1, comprising more concomitant compounds (such as phenols and flavone C-glycosides).

UVA and UVB light transmission (T %) was studied for different dilutions of Extract 2 (obtained according to Example 1 herein) with myconoside concentrations of 0.11 mg/ml, 0.055 mg/ml, 0.011 mg/ml, 0 0.0055 mg/ml, and 0.00275 mg/ml, as presented in FIG. 3.

The results indicate that a minimum concentration of 0.011 mg/ml of myconoside is sufficient to provide 50% inhibition of UV radiation transmission, and a maximum concentration of 0.11 mg/ml provides 100% protection. The results demonstrate the high potential of the phenylethanoid fraction (mainly myconoside and paucifloside) contained in Extracts 1 and 2 of the present invention to be used as a UV filter in sun protection products.

### Example 5

Investigation of the Antioxidant Potential of Extracts from In Vitro Systems of Haberlea rhodopensis, Obtained According to Example 1 Alone and in Combinations with Proven UV Protectors

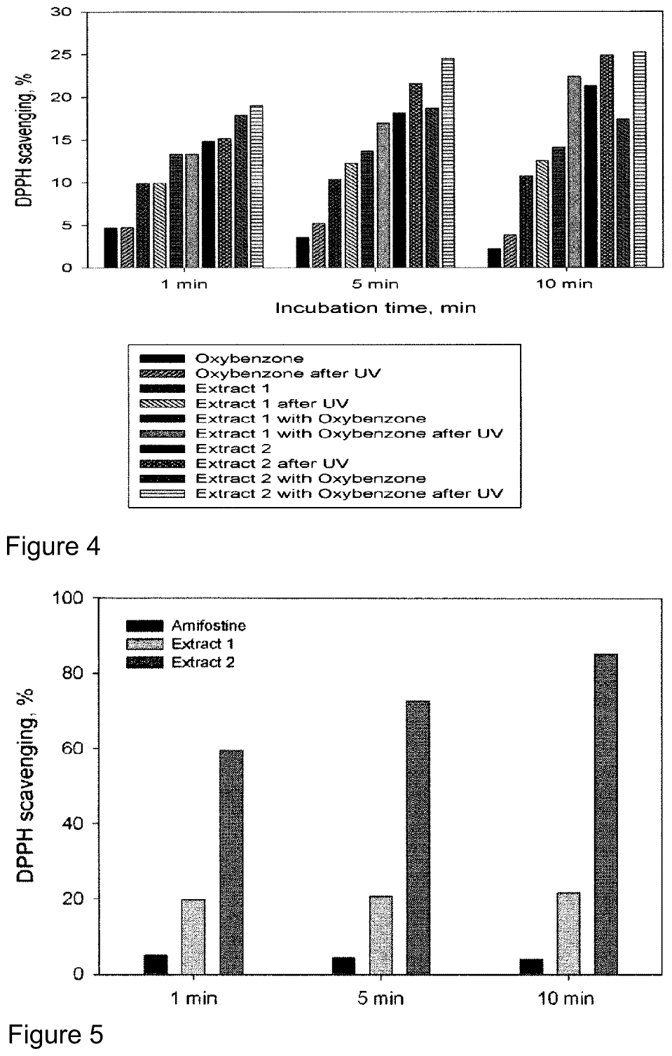

The radical scavenging activities (% inhibition) of aqueous solutions (0.5%) of Extract 1, Extract 2 (obtained according to Example 1), Oxybenzone, and 1:1 extract: Oxybenzon combinations (w/w) was evaluated by a DPPH test. A comparative DPPH analysis was carried out and the antioxidant potential of Extract 1, Extract 2, a commercial UV protector (Oxybenzone), as well as combinations of Extract 1 with Oxybenzone and of Extract 2 with Oxybenzone was compared. The results proved that myconoside from the purified Extract 2 is a carrier of antioxidant activity. The method of direct EPR spectroscopy was used (Yordanov and Christova, 1997; Karamalakova, 2014). To measure DPPH radical scavenging capacity, 98% ethanolic solution of DPPH (80 mM, stock) was used, room temperature (22° C.), and Extract 1, Extract 2, Oxybenzone and the relevant combinations (at a concentration of 0.5%) were mixed and homogenized. The mixture was incubated in the dark, and examined for DPPH-H/R radicals generated in the system at the 1^{st}, 5^{th} and 10^{th} minute. DPPH solution was used as an internal standard for the EPR signal (FIG. 4).

The results of the study found that there was a statistically significant increase in the percentage of trapped DPPH radicals in Extract 2 (from 14.9% to 18.2%, 21.3%, p<0.001), with the UV protector Oxybenzone showing the lowest radical scavenging activity (from 4.7% to 3.2% to 2.2%, p<0.001) (FIG. 4). Prolonged incubation of the investigated solutions did not bring about statistically significant change in the percentage of trapped DPPH radicals. The results indicate that Oxybenzone alone exhibits very low antioxidant activity, but when combined with Extract 1 and with Extract 2, it increases significantly (p<0.001). Induction of UV-B stress not only did not decrease inhibitory capacity, but also brought about a sharp, statistically significant increase thereof. This becomes visible after 10 min in both combinations. The conclusion is that in an in vitro environment, the studied extracts exhibit strong antioxidant activity and long-lasting free radical scavenging ability at different incubation times, prior to and following induced UV-B stress. This effect was most pronounced with purified Extract 2 (mainly myconoside and paucifloside) and its combination with the commercial UV protector Oxybenzone.

### Example 6

Investigation of the Antioxidant Potential of Extracts from In Vitro Systems of Haberlea rhodopensis, Obtained According to Example 1 Alone and in Combinations with Proven Radioprotectors

It has been proven that free radicals generated after exposure to gamma radiation are highly reactive and can trigger chain reactions leading to significant biological damage and increased oxidative stress. Hence, the so-called radioprotectors are of special interest for practical applications. They are substances and compounds of synthetic or natural origin that allow the body's cells to withstand higher levels of radiation or can quickly eliminate the harmful genotoxic effects of such radiation and maintain human genome stability. In order to prove that the phenylethanoid fraction (containing mainly myconoside and paucifloside) is a carrier of antioxidant activity and that it is also the main fraction responsible for the radioprotective properties of the extracts from in vitro systems of plants belonging to the family Gesneriaceae, in particular to the genera Haberlea and Ramonda, as well as their hybrids, we conducted comparative studies of the antioxidant activity of Extract 1, Extract 2, the commercial radioprotector amifostine, as well as combinations of Extract 1 with amifostine and Extract 2 with amifostine, by direct EPR spectroscopy. To measure the DPPH radical scavenging capacity, 98% ethanolic solution of DPPH (80 mM, stock) was used, room temperature (22° C.) and Extract 1, Extract 2, amifostine and the corresponding combinations (at a concentration of 0.5%) were mixed and homogenized. The mixture was incubated in the dark and examined for DPPH-H/R radicals generated in the system and at the 1^{st}, 5^{th} and 10^{th} minute. A DPPH solution was used as an internal standard for the EPR signal, and the DPPH radical scavenging activity (% inhibition) of aqueous solutions (0.5%) of Extract 1, Extract 2 (obtained according to Example 1) and amifostine was tested. (FIG. 5).

The results of the study showed a statistically significant increase in the percentage of trapped DPPH radicals in the purified Extract 2 (from 59.5% to 72.7%, 85.32%, p<0.001), while the commercial radioprotector amifostine showed the lowest radical scavenging activity (from 5.1% to 4.5%, 3.9%, p<0.001) (FIG. 5). It was found that prolonged incubation of the investigated solutions did not change the percentage of trapped DPPH radicals.

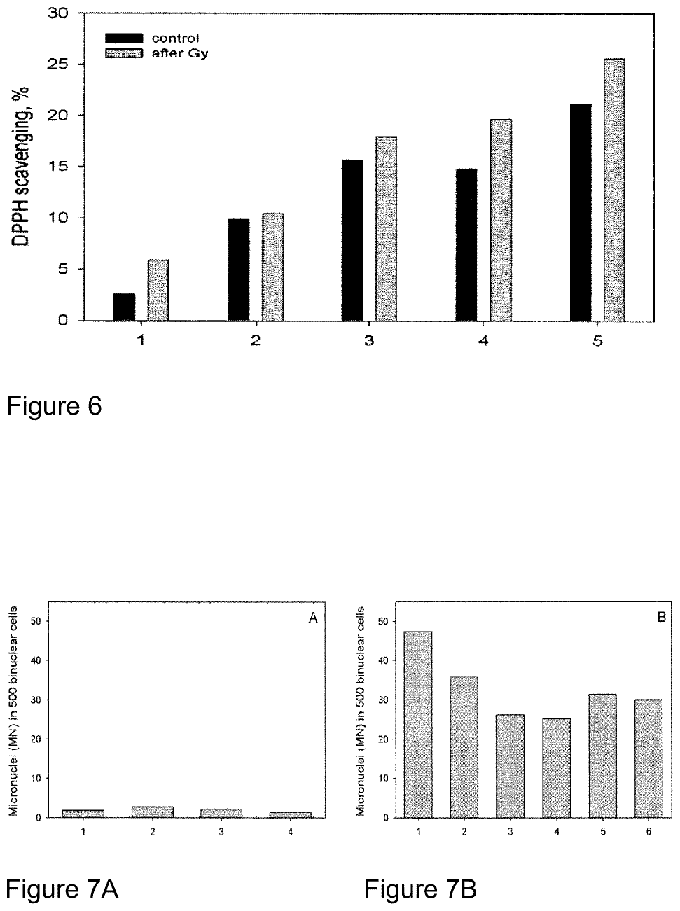

The effect of combinations of the investigated extracts with the commercial radioprotector amifostine was also studied before and after gamma irradiation (FIG. 6). 2.0 Gy 60Co gamma radiation (y-rays) was performed in a water bath (37° C.) with a Rokus-M irradiator at 24 Gy/min. DPPH radical scavenging activity (% inhibition) of aqueous solutions (0.25%) of amifostine (1), Extract 1 (2), 1:1 Extract 1:amifostine (w/w) (3), Extract 2 (obtained according to Example 1) (4), and 1:1 Extract 2:amifostine (w/w) (5) was measured before and after exposure to 2.0 Gy gamma radiation.

The results show a statistically significant increase in the percentage of trapped DPPH radicals after Gy irradiation in the purified Extract 2 (mainly containing myconoside and paucifloside) (19.7%, p<0.001), as well as in its combination with amifostine (25.6%, p<0.001). Prolonged (30 min) incubation of the studied solutions did not change the percentage of trapped DPPH radicals after exposure to 2.0 Gy irradiation. Amifostine (0.25%) alone showed insignificant antioxidant activity (2.5% before and 5.9% after gamma irradiation, p<0.001), but in combination with purified Extract 2 it increased significantly (21.1% before and 25.6% after gamma irradiation, p<0.005).

### Example 7

Investigation of Clastogenic Activity and Anticlastogenic Potential of Extracts from In Vitro Systems of Haberlea rhodopensis Derived According to Example 1, Alone and in Combination with Clinically Proven Cytoprotective Agents (Amifostine), Directly Added to Cell Cultures, and 1 Hour after Irradiation of Blood

The anticlastogenic potential of Extract 1 and Extract 2 (obtained according to Example 1), as well as of the clinically proven cytoprotective agent amifostine, was investigated individually and in combinations on human lymphocyte cultures in vitro before and after exposure to ionizing radiation (gamma rays) by micronucleus scoring tests. The micronucleus test of peripheral blood lymphocytes and polychromatic erythrocytes is the most appropriate test in screening for mutagenicity. A compound is considered mutagenic when it increases the spontaneous frequency of the micronuclei (MN) in polychromatic erythrocytes or in lymphocytes. This test demonstrates the mutagenic effect of compounds that are clastogens or spindle inhibitors. The cytokinesis-block micronucleus assay of Fenech, M. and A. Morley (1985) was used to analyze MN in binuclear lymphocytes from peripheral blood. The number of MN found in the examined 500 binuclear cells from cell cultures prepared with irradiated and non-irradiated peripheral venous blood taken from 5 healthy volunteers was investigated. The blood of the donors was exposed to 2.0 Gy 60Co gamma radiation (y-rays) in a water bath (37° C.) with a Rokus-M irradiator at 24 Gy/min. Exposure was calculated depending on the geometrical parameters of the irradiator, the distance and power of the source. FIG. 7 shows the number of MN found in the examination of 500 binuclear cells in the cell cultures prepared with non-irradiated blood (A) and irradiated blood (B) after the addition of: Control (1); Amifostine (2); Extract 1 (3); Extract 2 (4); 1:1 Extract 1:amifostine (w/w) (5), and 1:1 Extract 2:amifostine (w/w) (6).

The results from the addition of amifostine, Extract 1 and Extract 2 (obtained according to Example 1) to non-irradiated blood cells showed a lack of clastogenic activity of the tested substances, which is evidence that they are not mutagenic and can be safely used in medicine (FIG. 7A). The comparative statistical analysis of the results obtained for the total number of MN in 500 binuclear cells in different groups of cell cultures treated after irradiation with gamma radiation (FIG. 7B) revealed statistically significant differences between the control and the cells treated with Extract 1, Extract 2, as well as their combinations with amifostine (p>0.05).

### Example 8

Investigation of Clastogenic Activity and Anticlastogenic Potential of Extracts from In Vitro Systems of Haberlea rhodopensis Derived According to Example 1 Alone and in Combination with Clinically Proven Cytoprotective Agents (Amifostine) Added to Cell Cultures 1 Hour Before Irradiation with Ionizing Radiation (y-Rays) In Vitro

The clastogenic activity and anticlastogenic potential of Extract 1 and Extract 2 (obtained according to Example 1), as well as of the clinically proven cytoprotective agent amifostine, were investigated alone and in combination on human lymphocyte cultures in vitro before and after exposure to ionizing radiation (y-rays) through tests for detection of induced chromosomal aberrations. The method used enables accurate identification of all major types of structural chromosomal rearrangements induced by ionizing radiation (y-rays), such as single or double acentric fragments, dicentric chromosomes, and ring chromosomes.

The study design included a total of 6 groups, including the untreated control. In each group, 5 blood donors were examined, and in each donor 100 metaphase plates obtained from lymphocyte cells from peripheral venous blood were analyzed for chromosomal aberrations, or a total of 500 metaphase plates for each group.

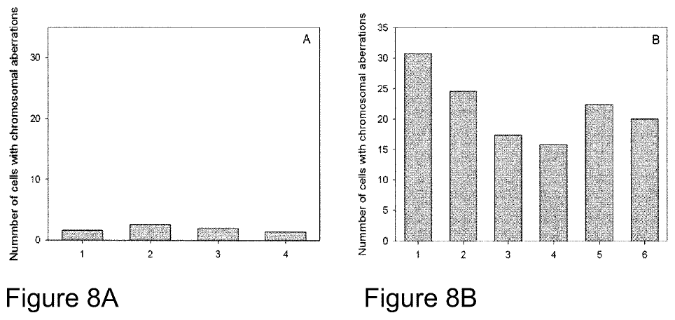

Cell cultures were prepared using the method of Evans H. J. for short-term culturing of peripheral blood lymphocytes and obtaining metaphase plates for chromosome aberration reading. Chromosome microscope slides were observed on an Olympus BX41 microscope at the most convenient magnifications—screening preparations at magnification 63 and detailed analysis of chromosomes for aberrations at magnification 1000. A minimum of 100 metaphases from each sample were analyzed. Donor cell cultures were exposed to 2.0 Gy 60Co gamma radiation (y-rays) in a water bath (37° C.) with a Rokus-M irradiator at 24 Gy/min. Exposure was calculated depending on the geometrical parameters of the irradiator, the distance and power of the source. FIG. 8 shows the frequency of aberrant cells in treated human lymphocytes before (A) and after (B) 2.0 Gy gamma irradiation as follows: Control (1); Amifostine (2); Extract 1 (3); Extract 2 (4); 1:1 Extract 1:amifostine (w/w) (5), and 1:1 Extract 2:amifostine (w/w) (6).

The results from the addition of amifostine, Extract 1 and Extract 2 (obtained according to Example 1) to non-irradiated blood cells showed no clastogenic activity of the substances tested, which is evidence that they are not mutagenic and are safe to use (FIG. 8A). The lack of clastogenic activity of the studied extracts, at the concentrations indicated in the experiments, gives grounds for Extract 1 and Extract 2, obtained according to the invention, to be used in phytotherapy and practical medicine. The comparative statistical analysis of the results obtained using pretreated cells before gamma irradiation (FIG. 8B) shows the presence of statistically significant differences with the control cells pretreated with Extract 1 and Extract 2 (p>0.01), as well as with their combinations with amifostine (p>0.05).

The results illustrated with Example 7 and Example 8 point out that the phenylethanoid-rich (mainly myconoside and paucifloside) extracts obtained according to Example 1 have a more pronounced anticlastogenic potential compared to synthetic amifostine, which is used as a radioprotector in clinical practice but has numerous side effects. Based on the absence of any cytotoxic and clastogenic activities of the extracts from in vitro systems of plants belonging to the family Gesneriaceae, in particular to the genera Haberlea and Ramonda, as well as their hybrids, it can reasonably be argued that they can be used in phytotherapy as radioprotectors due to their strong anticlastogenic potential.

According to Examples 4-8, similar parallel tests were also carried out for an extract of Ramonda serbica obtained according to Example 3a, and the results were close to those presented, demonstrating a deviation from ±0.1% to +0.9%.

Similar parallel tests were conducted for a combination of Extract 1 and Extract 2 from Haberlea rhodopensis Friv. in a ratio of 1:1, obtained according to Example 1, with the results obtained being close to those presented and demonstrate a deviation from ±0.05% to ±0.09%.

1-10. (canceled)

11. A myconoside containing extract from in vitro culture system of plants belonging to the family Gesneriaceae, wherein the extract comprises a phenolic fraction (Extract 1) comprising flavone-C glycosides and phenylethanoids.

12. A myconoside containing extract from in vitro culture systems according to claim 11, wherein the phenylethanoids comprise 0.1 to 55.0 wt % myconoside and paucifloside.

13. A myconoside containing extract from in vitro culture systems according to claim 11, wherein the extract further comprises a phenylethanoids fraction (Extract 2) comprising 55.0 to 99.9 wt % myconoside and paucifloside.

14. A myconoside containing extract from in vitro culture systems according to claim 11, wherein the plant family Gesneriaceae comprises the genera Haberlea and Ramonda.

15. A myconoside containing extract from in vitro culture systems according to claim 14, wherein the plant is selected from the species Haberlea rhodopensis Friv., Ramonda heldreichii (Boiss.) C. B. Clarke, Ramonda myconi (L.) Rchb.; Ramonda nathaliae Pancic & Petrovic, and Ramonda serbica Pancic.

16. A myconoside containing extract from in vitro culture systems according to claim 13, wherein the ratio in the Extract 1:Extract 2 combination varies from 1:99 to 99:1.

17. A composition comprising a myconoside containing extract from in vitro culture systems according to claim 11, wherein the extract is conditioned with a solvent suitable for food, pharmaceutical and cosmetic purposes, such as water, glycerin, propylene glycol or butyl glycol.

18. A composition comprising a myconoside containing extract from in vitro culture systems according to claim 13, with a final standardised concentration of myconoside of 0.001% to 99% when Extract 1 and Extract 2 are diluted separately, or when combined in different ratios.

19. Method of preparation of a myconoside containing extract from in vitro culture system of plants belonging to the family Gesneriaceae, the method including the following sequential steps:
a) the biomass obtained after initiation and propagation of the in vitro system is subjected to extraction with water or a water-alcohol mixture (40-90%);
b) the purified aqueous phase from step a) is concentrated under vacuum at a temperature of 40° C.-60° C. to remove the solvent until a crude extract is obtained and the crude extract is subjected to solid-phase extraction with a hydrophobic-interacting resin;
c) elution of the phenolic fraction from the resin with a mobile phase chosen from solvent of different polarity to obtain Extract 1 comprising 0.1 to 55.0 wt % of phenylethanoid glycosides; and optionally
d) elution of the phenylethanoid fraction from the resin with a water-alcohol mixture of C1-C3 alcohols and distilled water 5 to 70% v/v to obtain Extract 2 comprising 55.0 to 99.9 wt % of phenylethanoid glycosides;
wherein the phenylethanoid glycosides comprise myconoside and paucifloside.

20. A method according to claim 19, wherein the in vitro culture system is selected from the species Haberlea rhodopensis Friv., Ramonda heldreichii (Boiss.) C. B. Clarke, Ramonda myconi (L.) Rchb.; Ramonda nathaliae Pancic & Petrovic, and Ramonda serbica Pancic.

21. A method according to claim 19, wherein the hydrophobic-interacting resin comprises C18 reversed-phase resin.

22. A method according to claim 19, wherein the solvent of different polarity is selected from ethyl acetate or isopropyl alcohol.

23. A method according to claim 19, wherein the phenylethanoid glycosides comprise myconoside and paucifloside.

24. A myconoside containing extract obtained by the method of claim 19.

25. A combination of myconoside containing extract from in vitro culture systems according to claim 11 and a safe agent selected from a UV protector, chemoprotective, radioprotector or cytoprotective agent.

26. A combination according to claim 25, wherein the safe agent is selected from oxybenzone or amifostine.

27. A product comprising a myconoside containing extract according to claim 11.
